SEQUENCE LISTING (2 sequences)

SEQ ID NO: 1 - protein, 10 aa, Homo sapiens
  MOD_RES (1)..(1): Glu is pyroglutamate
EHWSYGLRPG

SEQ ID NO: 2 - DNA, 60 bp, artificial sequence
  GnRH-binding nucleic acid sequence
gcggcggagggtgggctggggctgggccggggggcgtgcgtaagcacgtagcctcgccgc

(19) United States
(12) Patent Application Publication
(10) Pub. No.: US 2005/0208487 A1
(43) Pub. Date: Sep. 22, 2005

(54) Immobilized nuckeic acids and uses thereof

(76) Inventors: Jens Burmeister, Koln (DE); Petra Burgstaller, Planegg (DE); Sven Klussmann, Berlin (DE); Thomas Klein, Potsdam (DE); Christian Frauendorf, Berlin (DE)

Correspondence Address: DLA PIPER RUDNICK GRAY CARY US, LLP, 1625 MASSACHUSETTS AVENUE, NW, SUITE 300, WASHINGTON, DC, 20036-2247, US

(21) Appl. No.: 10/296,506

(22) PCT Filed: May 25, 2001
(86) PCT No.: PCT/EP01/06014

§ 371 Date: May 21, 2003

(30) Foreign Application Priority Data
May 26, 2000 (DE) 100 26 300.3

(51) Int. Cl. C12Q 1/68; C07H 21/04

(52) U.S. Cl. 435/6; 536/25.4

(57) ABSTRACT

One aspect of the present invention relates to an immobilized nucleic acid comprising a nucleic acid and a matrix where the nucleic acid is a Spiegelmer and the Spiegelmer is functionally active. Another aspect of the invention concerns an immobilized nucleic acid comprising a nucleic acid and a matrix where the nucleic acid is coupled to the matrix at least via its 3′ end and the nucleic acid is a functional nucleic acid. Finally a further aspect of the invention concerns the use of such immobilized nucleic acids as affinity ligands for example in chromatography and in apheresis.

The present invention is related to immobilized nucleic acids, their use in apheresis and affinity purification, an apheresis device containing these immobilized nucleic acids and methods for the production thereof.

Apheresis or plasmapheresis is, on the one hand, a preparative method for isolating donor plasma and certain blood cells and, on the other hand, a therapeutic method in which specific plasma components are removed. Apheresis is for example used as LDL apheresis in familial hypercholesterolemia, lipidapheresis, immunoapheresis to remove autoantibodies and cytoapheresis to separate erythrocytes or leucocytes.

The object especially of therapeutic apheresis is in general to bind undesired molecules from the blood to an adsorber column outside the body in order to improve a particular clinical picture. The advantage of apheresis compared to administering active substances to the organism is that fewer side effects occur.

Examples of functional ligands used in apheresis and also for affinity purification are antibodies, proteins and peptides which are immobilized specifically or unspecifically on support materials and have already been used for many years for apheresis. The adsorber column can then be connected to a plasma separation machine for plasmapheresis or it can be incorporated directly into the extracorporeal bloodstream of a patient in the case of whole blood cleansing using a suitable additional solid phase. Blood is passed over the adsorber column in such a manner that the harmful substances and molecules are retained on the adsorber column by interaction with the immobilized ligands. The serum or blood purified in this manner is then returned to the body of the patient.

The problems in developing apheresis systems are to find and produce a suitable affinity ligand that can be immobilized in its native state, i.e. while retaining its relevant binding characteristics, on a (usually) solid phase such that it remains functional during and after the production process. It should be preferably possible to sterilize the ligand ideally by steam sterilization. Moreover an important property of the ligand must be its stability in a serum or whole blood environment i.e. it must have a sufficiently long half-life under the apheresis conditions towards degradative enzymes in the serum or blood.

The object of the present invention is to find a ligand that can be used in particular in apheresis and which meets the above-mentioned requirements. Another object is to provide an affinity system and in particular an apheresis system which allows a highly specific removal of certain substances present in a fluid and in particular blood or serum and, at the same time, does not have the above-mentioned disadvantages and shortcomings of the affinity ligands of the apheresis systems known in the prior art.

Another object of the present invention is to provide a method which allows the dissolution of a complex of functional nucleic acid and target molecule especially when the functional nucleic acid is bound to or immobilized on a matrix or a solid support which is used synonymously herein.

The object is achieved according to the invention by an immobilized nucleic acid comprising a nucleic acid and a matrix wherein the nucleic acid is a Spiegelmer and the Spiegelmer is functionally active.

One embodiment provides that the 3′ end of the Spiegelmer is bound to the matrix.

Another embodiment provides that the 5′ end of the Spiegelmer is bound to the matrix.

According to the invention the object is also achieved by an immobilized nucleic acid comprising a nucleic acid and a matrix wherein the 3′ end of the nucleic acid is bound to the matrix. It is particularly preferred when the nucleic acid is a functional nucleic acid.

In addition the object is achieved according to the invention by the use of the immobilized nucleic acids according to the invention as an affinity medium and in particular as an affinity medium in affinity purification and preferably in affinity chromatography.

In a further aspect the object is achieved by use of the immobilized nucleic acid according to the invention for apheresis i.e. for extracorporeal blood cleansing.

The object is also achieved by an apheresis device which contains the immobilized nucleic acid according to the invention.

Finally the object is achieved by a method for producing an immobilized nucleic acid in particular for producing the immobilized nucleic acid according to the invention which comprises the following steps:
providing a nucleic acid and a matrix and
reacting the nucleic acid and the matrix to form a bond between the 3′ end and/or the 5′ end of the nucleic acid and the matrix.

The object is alternatively also achieved by a method which comprises the following steps:
providing a nucleic acid and a matrix and
reacting the nucleic acid and the matrix to form a bond between the 3′ end and 5′ end of the nucleic acid and the matrix.

Furthermore the object is achieved according to the invention by a method for eluting a target molecule bound to a nucleic acid, in particular to an immobilized nucleic acid according to the invention, wherein it is eluted using distilled water at an elevated temperature. The elevated temperature is preferably at least 45° C., preferably at least 50° C. and more preferably at least 55° C.

In yet another aspect the object is achieved by a method for eluting a target molecule bound to an immobilized nucleic acid according to the invention wherein the elution is a denaturing elution.

One embodiment provides that the denaturing elution uses a compound selected from the group comprising guanidinium thiocyanate, urea, guanidinium hydrochloride, ethylene diamine tetraacetate, sodium hydroxide and potassium hydroxide.

In one embodiment of the immobilized nucleic acid according to the invention the functional nucleic acid is selected from the group comprising aptamers.

Another embodiment of the nucleic acid according to the invention provides that the immobilized nucleic acid, in addition to the binding via its 3′ end or its 5′ end, is bound via at least one other site to the matrix. The other site is preferably the 5′ end of the nucleic acid. Alternatively or in addition the other site may be a site within the sequence of the nucleic acid. In another preferred embodiment the nucleic acid, in addition to being bound to the matrix via its 3′ end, is also bound via its 5′ end and additionally via at least one site within the sequence of the nucleic acid.

In a preferred embodiment the immobilized nucleic acids according to the invention are modified at their 5′ end.

In another preferred embodiment the nucleic acid contains nucleotides which are selected from the group comprising D-nucleotides, L-nucleotides, modified D-nucleotides and modified L-nucleotides and mixtures thereof.

In an embodiment of the immobilized nucleic acid according to the invention the nucleic acid is directly bound to the matrix.

In an alternative embodiment of the immobilized nucleic acid the nucleic acid is bound to the matrix by means of a linker structure.

In an embodiment of the immobilized nucleic acid according to the invention the linker structure is bound to the 3′ end as well as to the 5′ end of the nucleic acid and the linker structure is also bound to the matrix; this results in the formation of a Y-shaped linker structure.

In another embodiment the linker structure is a spacer wherein the spacer preferably comprises at least four atoms which are selected from the group comprising C atoms and heteroatoms.

In yet another embodiment the structure of the nucleic acid used to directly or indirectly bind the nucleic acid to the matrix is selected from the group comprising the sugar moiety of the sugar phosphate backbone, the phosphate moiety of the sugar phosphate backbone and the base moiety of the nucleotides forming the nucleic acid.

The binding of the immobilized nucleic acid according to the invention can be selected from the group comprising covalent bonds, non-covalent bonds, especially hydrogen bonds, van der Waals interactions, coulombic interactions and/or hydrophobic interactions, coordinate bonds and combinations thereof.

In addition one embodiment provides that the matrix is a solid phase or a solid support.

In a preferred embodiment of the immobilized nucleic acid the solid phase, matrix or solid support comprises a material which is selected from the group comprising organic and inorganic polymers.

Another embodiment of the immobilized nucleic acid according to the invention provides that the nucleic acid has a minimum length wherein the minimum length is selected from the group comprising the minimum lengths of about 15 nucleotides, 20 nucleotides, 25 nucleotides, 30 nucleotides, 35 nucleotides, 40 nucleotides, 50 nucleotides, 60 nucleotides, 90 nucleotides and 100 nucleotides.

Another embodiment provides that the matrix is selected from the group comprising CPG, sepharose, agarose, Eupergit and polystyrene.

Another embodiment provides that the immobilized nucleic acid contains a nucleic acid sequence according to SEQ ID No. 2.

The method according to the invention and in particular in the method according to the invention in which the nucleic acid and the matrix are reacted to form a bond between the 3′ end of the nucleic acid and the matrix may additionally comprise the step:
modifying the 5′ end of the nucleic acid before the reaction.

Furthermore the method according to the invention and in particular the method according to the invention in which the nucleic acid and the matrix are reacted to form a bond between the 3′ end of the nucleic acid and the matrix may additionally comprise the following step:
modifying the 5′ end of the nucleic acid after the reaction.

Finally the nucleic acid and/or the matrix may be activated before reacting the nucleic acid and the matrix in the method according to the invention.

Furthermore in the method according to the invention the nucleic acid and/or the matrix may be provided before or during the reaction with a linker structure or a part thereof or a spacer. This also applies to the case in which the nucleic acid is bound to the matrix by means of a Y-shaped spacer or a Y-shaped linker structure. In this case it may be advantageous to firstly bind the 3′ and the 5′ end of the nucleic acid to the linker structure or a to a part thereof and subsequently bind this to the matrix. However, within the scope of the invention it is also possible to firstly bind the linker structure or a part thereof to the matrix and subsequently bind the 3′ end and the 5′ end of the nucleic acid to this. Finally intermediary stages of the two basic methods are possible i.e. binding one end of the nucleic acid to the linker structure or to a part thereof, then binding a complex formed in this manner to the matrix and finally binding the end of the nucleic acid which has not yet bound to the linker structure or to a part thereof, to the linker structure or to a part thereof.

The basis of the present invention is the surprising finding that binding or coupling of a nucleic acid to a matrix via the 3′ end of the nucleic acid whether directly or indirectly i.e. using a linker structure or a spacer, protects the bound or immobilized nucleic acid (also referred to herein as coupled nucleic acid) from enzymes such as nucleases (endonucleases and 3′ and 5′ exonucleases) and in particular 3′ modified enzymes, and also results in a stabilization especially towards elevated temperatures and pressures and thus stability under sterilization conditions and in particular under steam sterilization conditions. Stability as used herein also means the maintenance of the structure and function of the immobilized functional nucleic acid under the conditions of apheresis; this relates to the biological stability of the functional and immobilized nucleic acid towards degradative enzymes. Furthermore the immobilization of the nucleic acid also ensures that it is protected against unspecific hydrolysis during storage. This increased stability resulting from the binding of the nucleic acid via at least its 3′ end to the matrix compared to other types of immobilization is exhibited as an increased half-life which is defined as the extent to which a property of the nucleic acid changes over time. Such properties may be among others: amount of bound nucleic acid (for example per matrix surface), binding properties for target molecules and suchlike.

If the coupled nucleic acid is a functional nucleic acid, the effects described above also occur. In this case it is remarkable that not only the stability of the nucleic acid but also the functionality of the nucleic acid is retained by the type of immobilization or binding by means of the 3′ end of the nucleic acid even towards the enzyme activities listed above and under sterilization conditions and in particular under steam sterilization.

The ability of the immobilized nucleic acid to bind to a target molecule or an interaction partner, especially when it is a functional nucleic acid, is surprising since the binding properties of the functional nucleic acids are totally different from the known binding in the prior art of nucleic acids to other nucleic acids via base-base interactions. Binding between functional nucleic acids and their target or their target structure or their target molecule requires the formation of a distinct two-dimensional and three-dimensional structure and thus of binding pockets. Moreover it also appears that functional nucleic acids such as aptamers and Spiegelmers bind by means of the well-known induced fit mechanism (Westhof, E. & Patel, D. (1997) Curr Opin Struct Biol 7, 305-309) i.e. the structure of the functional nucleic acid present in solution is different from the structure of the functional nucleic acid in the complex of target molecule and functional nucleic acid. It was completely surprising that this folding mechanism of the functional nucleic acid which is a prerequisite for the formation of the said complex can also occur when the functional nucleic acid is immobilized.

The above-mentioned surprising properties of a nucleic acid bound or immobilized in such a manner are also observed when, in addition to the coupling via the 3′ end of the nucleic acid, the nucleic acid is also coupled via its 5′ end whereby, with regard to the coupling method, it is possible to firstly immobilize the 5′ end and then the 3′ end, or firstly immobilize the 3′ end and then the 5′ end or to immobilize both ends simultaneously (M. Kwiatkowski et al., Nucl. Acids Res. 1999, 27, 4710-4714). The same also applies to the situation in which, in addition to the binding of the nucleic acid to the matrix via the 3′ end of the nucleic acid and simultaneously via the 5′ end, at least one other site of the nucleic acid is used to bind it to the matrix. Such another site is one which is contained in the sequence of the nucleic acid i.e. the sequence of nucleotides. Hence within the scope of the present invention nucleotides within the immobilized nucleic acid or within the nucleic acid to be immobilized can be used to bind the nucleic acid to the matrix. As for the immobilization or binding of the nucleic acid by its 3′ end, it is also possible to use the sugar moiety of the sugar phosphate backbone, the phosphate moiety of the sugar phosphate backbone and/or the base moiety of nucleotides forming the nucleic acid in order to form the direct or indirect bond. Any of the aforementioned moieties may be present in a modified form and the modification may be carried out either for the purposes of immobilization or for the purpose of stabilizing the nucleic acid.

As a result of these surprising properties, such an immobilized nucleic acid is outstandingly suitable as an affinity ligand for apheresis and affinity chromatography especially when the nucleic acid is functional i.e. interacts specifically with a compound, molecule or molecular structure (or part thereof). The interaction may be reversible or irreversible, a reversible interaction being preferred since it allows a regeneration and thus the reuse of the coupled nucleic acid.

In this connection it is particularly noteworthy that it was discovered that the immobilized Spiegelmers are not only biologically and physically stable, but also that they retain their affinity and specificity for the target molecule. This is surprising in view of the fact that although the so-called Spiegelmer technology has only recently been developed starting from a nucleic acid sequence composed of naturally-occurring D-nucleotides which binds to a target molecule present in its unnatural form, for example a D-peptide, it is nevertheless possible to produce a nucleic acid having the same sequence but composed of unnatural L-nucleotides which then binds to the naturally-occurring target molecule i.e. to the L-protein in the case of a protein.

Functionality of a nucleic acid is understood herein to mean the intrinsic binding property or affinity of this nucleic acid for a target molecule (also referred to here as target or target molecule) which is due to non-covalent interactions such as hydrogen bonds, coulombic interactions, van der Waals interactions, hydrophobic interactions, coordinative interactions (either individual or combinations thereof) between the nucleic acid and the target. In individual cases the non-covalent interaction can also be converted into a covalent interaction.

“Functional nucleic acid” is understood herein to mean in particular a nucleic acid which is the result of the selection procedure described herein. Hence functional nucleic acids are in particular those which bind to a target molecule or to a part thereof and are the result of contacting a nucleic acid library in particular a statistical nucleic acid library with the target molecule. Hence functional nucleic acids are in particular also aptamers and Spiegelmers. The immobilized nucleic acids disclosed herein and in particular the immobilized aptamers and immobilized Spiegelmers are thus physically and biologically stable immobilized nucleic acids. As a result of this stability it is possible to use the immobilized aptamers as well as the immobilized Spiegelmers for apheresis. Functionally active nucleic acids are in particular those which bind to a target molecule or to a part thereof, preferably with high affinity and specificity and are in particular the result of contacting a nucleic acid library in particular a statistical nucleic acid library with the target molecule. Hence functional nucleic acids are in particular also aptamers and Spiegelmers.

Another application of the immobilized aptamers and the immobilized Spiegelmers is their use in affinity purification such as affinity chromatography. In apheresis as well as in affinity purification the aptamers and also the Spiegelmers represent the—biologically and physically—stable ligands, which are immobilized on suitable matrices while retaining the high affinity and specificity typical of functional nucleic acids towards the target molecule or a part thereof which has been conventionally used to generate them in evolutionary selection processes.

Suitable support materials and matrices are known to persons skilled in the art in this field for both applications which can all be basically also used to immobilize aptamers and Spiegelmers. The types of immobilization and immobilization conditions are known to experts in the field and include especially those that are described herein. In this connection it should be noted with reference to the examples that the described techniques can in principle be used for aptamers as well as Spiegelmers.

Within the scope of the present invention an aptamer and/or a Spiegelmer is preferably used as the nucleic acid which is bound to or immobilized on a matrix.

Aptamers are short oligonucleotides based on DNA or RNA which have a binding property for a target molecule; the DNA or RNA molecules can be composed of naturally configured as well as non-naturally configured nucleotides or mixtures thereof which according to the prior art may also have modified bases or sugars. Common modifications of sugars in nucleic acid molecules are for example 2′-amino, 2′-O-alkyl, 2′-O-allyl modifications (Osborne & Ellington, 1997, Chem. Rev. 97, 349-370). The sugar phosphate backbone may also be modified by using peptide nucleic acids (PNA) and other backbone modifications are described in R. S. Varma, 1993, SYNLETT September.

Combinatorial DNA libraries are firstly constructed for a selection method to develop functional nucleic acids. This usually involves the synthesis of DNA oligonucleotides which contain a central region of 10-100 randomized nucleotides which are flanked by two primer binding regions at the 5′ and 3′ terminus. The construction of such combinatorial libraries is described for example in Conrad, R. C., Giver, L., Tian, Y. and Ellington, A. D., 1996, Methods Enzymol., vol. 267, 336-367. Such a chemically synthesized single-stranded DNA library can be converted by means of the polymerase chain reaction into a double-stranded library which can be used as such for a selection. However, the single-strands are usually separated using suitable methods to obtain a single-stranded library which is used for the in vitro selection method when this is a DNA selection (Bock, L. C., Griffin, L. C., Latham, J. A., Vermaas, E. H. and Toole, J. J., 1992, Nature, vol. 355, 564-566). However, it is also possible to use the chemically synthesized DNA library directly in the in vitro selection. Moreover, it is also in principle possible to construct an RNA library from double-stranded DNA when a T7 promoter has been previously inserted, also by means of a suitable DNA-dependent polymerase e.g. T7 RNA polymerase. T7 RNA polymerase is also able to incorporate 2′-fluoro- or 2′-amino-nucleotides. The described methods can be used to construct libraries of 10¹⁵ and more DNA or RNA molecules. Each molecule from this library has a different sequence and hence a different three-dimensional structure. The in vitro selection method can now be used to isolate one or more DNA molecules from the said library by several cycles of selection and amplification and optionally mutation, where these DNA molecules have a significant binding property towards a specified target. The targets can for example be viruses, proteins, peptides, nucleic acids, small molecules such as metabolites of metabolism, pharmaceutical substances or metabolites thereof or other chemical, biochemical or biological components such as those described in Gold, L., Polisky, B., Uhlenbeck, O. and Yarus, 1995, Annu. Rev. Biochem. vol. 64, 763-797 and Lorsch, J. R. and Szostak, J. W., 1996, Combinatorial Libraries, Synthesis, Screening and application potential, ed. Riccardo Cortese, Walter de Gruyter, Berlin. The method is carried out in such a manner that binding DNA or RNA molecules are isolated from the original library and amplified after the selection step by means of the polymerase chain reaction. In the case of RNA selections a reverse transcription has to precede the amplification step by the polymerase chain reaction. A library enriched after a first selection round can then be used in a new selection round such that the molecules which accumulate in the first selection round have a chance to again prevail by selection and amplification and to enter a further selection round with even more daughter molecules. At the same time the polymerase chain reaction step allows new mutations to be introduced in the amplification e.g. by varying the salt concentration. After sufficient selection and amplification rounds, the binding molecules prevail. Hence an enriched pool has been formed whose members can be separated by cloning and subsequently the primary structures can be determined with conventional DNA sequencing methods. The binding properties of the sequences obtained are then examined with regard to the target. The method for generating such aptamers is also referred to as the SELEX method and is for example described in EP 0 533 838 the disclosure of which is herein incorporated by reference.

The best binding molecules can be shortened to leave only the essential binding domain by truncating the primary sequences and they can also be chemically or enzymatically synthesized.

So-called Spiegelmers are a special form of aptamers which are essentially characterized in that they are at least partially and preferably completely composed of unnatural L-nucleotides. Methods for producing such Spiegelmers are described in PCT/EP97/04726 the disclosure of which is hereby incorporated by reference. A characteristic feature of this method is the generation of enantiomeric nucleic acid molecules which bind to a native target i.e. a target present in its natural form or configuration or to such a target structure. The in vitro selection method described above is used to firstly select sequences that bind to the enantiomeric structure of a naturally occurring target. The sequences of the binding molecules (D-DNA, D-RNA or corresponding D-derivatives) obtained in this manner are determined and an identical sequence is then synthesized using mirror-image nucleotide building blocks (L-nucleotides or L-nucleotide derivatives). The mirror-image, enantiomeric nucleic acids (L-DNA, L-RNA or corresponding L-derivatives) obtained in this manner, the so-called Spiegelmers, have a mirror-image tertiary structure for symmetry reasons and thus have a binding property for a target which is present in its natural form or configuration.

In addition to the use of “pure” aptamers i.e. aptamers which are only composed of naturally occurring D-nucleotides or derivatives thereof, it is also possible that one or more of the nucleotides in the aptamer are in a non-natural form. Similarly the naturally-occurring and/or the non-naturally occurring nucleotides may be modified. Such modifications may be for example on the sugar phosphate backbone and on the nucleobases of the nucleic acid. The aforementioned for aptamers also applies to Spiegelmers.

There are in principle no limitations to the length of the immobilized nucleic acid. However, the immobilized nucleic acid preferably consists of at least 25 nucleotides. Other preferred mininium lengths of the immobilized nucleic acid are lengths of at least 15 nucleotides, 20 nucleotides, 25 nucleotides, 30 nucleotides, 35 nucleotides, 40 nucleotides, 50 nucleotides, 60 nucleotides, 90 nucleotides and 100 nucleotides.

In the method for eluting a target molecule bound to a nucleic acid in particular to an immobilized nucleic acid according to the invention, the elution is carried out at an. elevated temperature using distilled water. Surprisingly this simple method dissociates the complex of immobilized nucleic acid and target molecule without having to use salts which are often required for normal elution methods. This has advantages especially for industrial applications of the immobilized nucleic acid in that the salt load in the waste water is correspondingly low or if chemicals other than salts are used for the elution it is not necessary to use the corresponding compounds such as urea and guanidinium thiocyanate. Even under these elution conditions it is still possible to reuse the immobilized nucleic acid as an affinity matrix. The operating temperatures can be easily determined by a routine check starting with the specific nucleic acid target molecule pair. The temperature is typically at least 45° C. Other preferred temperature ranges are at least 50° C. or 55° C.

The discovery which forms the basis of this method is surprising since it has previously been assumed that salt solutions of high molarity are required to dissociate the specific interactions between the nucleic acid and target molecule. These salts or mechanisms which eliminate interference or dissociate complexes are not practical when using deionized, distilled or additionally purified water for the elution within the scope of the invention. Rather it appears to be the case that in the absence of such stabilizing salts or compounds when using desalted water, the structure either of the target molecule or also of the immobilized nucleic acid is changed to such an extent that the complex of immobilized nucleic acid and target molecule dissociates.

A modification of the sugar phosphate backbone or of the nucleobase(s) can, in addition to improving the stability of the coupled nucleic acid, also be used for coupling or immobilization while retaining the functionality of the nucleic acid. In this connection it is also possible to functionalise at least one of the bases of the nucleic acids which is/are not essential for the function of the nucleic acid such that the coupling or immobilization can occur while retaining the function.

The chemical synthesis of DNA molecules and RNA molecules required for the preparation of immobilized nucleic acids has been well established for more than 20 years and can be carried out in good yields e.g. by the phosphoramidite method (Beaucage & Iyer 1992, Tetrahedron Lett. 22, 1859-1862). Other synthesis strategies such as the H-phosphonate or the phosphoric acid triester method are described in Blackburn, G. M. and Gait, M. J., 1992, Nucleic acids in Chemistry and Biology, IRL Press Oxford and in Marshall & Boymel (Drug Discovery Today, 1998, vol. 3, No. 1) the disclosure of which is herewith incorporated by reference. Chemical synthesis can also be used as a simple method for the introduction of modifications as well as for the purposes of immobilization and stabilization of the nucleic acid in RNA and DNA molecules. The termini i.e. ends as well as the phosphate backbone can be chemically modified with various reagents. Modifications can be attached to the nucleic acid during the solid phase synthesis as well as after the synthesis. An example of a linker molecule inserted during the chemical synthesis is (1-dimethoxy-trityloxy-3-fluorenylmethoxycarbonylamino-hexane-2-methylsuccinoyl)-long chain alkylamino-CPG (3′-amino modifier C7 CPG, Glen Research, Virginia, USA). This linker molecule or this linker structure is used to attach a spacer consisting of 7 atoms which ends with a primary amino group to the 3′ phosphate of the nucleic acid. A second example is the 3′ terminal introduction of a spacer linked to biotin by using 1-dimethoxytrityloxy-3-O—(N-biotinyl-3-aminopropyl)-triethyleneglycol-glyceryl-2-O-succinoyl long chain alkylamino CPG (biotin TEG, CPG, Glen Research, Virginia, USA). A review of possible modifications which can be introduced during the synthesis is given in the product information of Glen Research, Virginia, USA: User Guide to DNA Modification, Products for DNA Research, and S. L. Beaucage, R. P. Iyer, Tetrahedron 1993, 49, 1925-1963).

The DNA can for example be postsynthetically modified by means of homo- or heterobifunctional linker molecules which are either already pre-activated or activated by adding suitable coupling reagents. Examples of non-activated homobifunctional linker molecules are diamines or dicarboxylic acids, examples of activated homobifimctional linkers are glutardialdehyde or the anhydrides of dicarboxylic acids, an N-hydroxysuccinimide ester activated with pyridyl disulfide being an example of a pre-activated heterobifunctional linker etc.

It has been shown that it is possible to develop functional aptamers and Spiegelmers that are outstandingly suitable as specific ligands for the development of completely novel adsorbers for extracorporeal blood purification. For this purpose aptamers or Spiegelmers are produced using the in vitro selection or in vitro evolution methods described above which are directed against molecules or structures which are referred to as the target and may for example be responsible for the development of one or more diseases. Said molecules or structures can for example be viruses, viroids, bacteria, cell surfaces, cell organelles, proteins, peptides, nucleic acids, small molecules such as metabolites, pharmaceutical substances or metabolites thereof or other chemical, biochemical or biological components as targets. The aptamers are characterized according to their properties. Aptamers in their native, i.e. non-derivatized form are usually unsuitable for therapeutic apheresis since they are degraded by nucleases in a biological fluid environment such as blood and thus lose their functionality. However, it surprisingly turned out that the nucleic acids according to the invention i.e. coupled nucleic acids in which the nucleic acid is coupled to a matrix via at least its 3′ end and hence coupled to a suitable solid phase for apheresis via its 3′ end by immobilizing the aptamer according to the invention, in particular using an additional modification at the 5′ end or by a double immobilization via the 3′ and the 5′ end, can result in an extremely high stability in human serum and whole blood.

The above-mentioned for aptamers also applies in the same sense to Spiegelmers. The term binding of a nucleic acid to a matrix is understood herein to mean that the nucleic acid is directly or indirectly bound to the matrix by means of various types of binding. The various types of binding include among others covalent binding, non-covalent binding (in particular hydrogen bonds, coulombic interaction, van der Waals interactions, hydrophobic interaction and ionic binding) and coordinative binding as further defined herein in connection with the various types of immobilization. The term binding as used herein also encompasses the term immobilization i.e. binding a compound to a support. The support does not have to be present as a solid phase; although a solid phase is preferred as a support.

Solid phases which can be solid or porous materials are also particularly suitable as matrices for binding or immobilizing nucleic acids. Such matrices are described for example in P. D. G. Dean, W. S. Johnson, F. A. Middle (Ed.), Affinity Chromatography-a practical approach, IRL Press, Oxford, 1985. The following matrices are mentioned as examples: agarose (a linear polymer isolated from red algae, composed of alternating D-galactose and 3,6-anhydrol-L-galactose residues), porous, particulate clay (aluminium oxide), cellulose (linear polymer of β-1,4-linked D-glucose with some 1,6 linkages), dextran (high molecular glucose polymer), Eupergit™ (Röhm Pharma, oxirane-derivatized acrylic beads; copolymer of methacrylamide, methylene-bis-acrylamide, glycidyl-methacrylate and/or allyl-glycidyl ether. From: product information of the Röhm Company), glass, controlled pore glass (CPG), is manufactured by heating borosilicate glasses for a long period at 500-800° C. After phase separation the borate-rich phases are dissolved away under acidic conditions to form tunnels and pores of 25-70 Angstroms in size. The glass surface is usually derivatized with silane-containing compounds. From: Affinity Chromatography-a practical approach, IRL Press, Oxford, 1985), hydroxyalkyl methacrylate, polyacrylamide, Sephadex™ (dextran-based gel. From: product information of Amersham Pharmacia Biotech), Sepharose, Superose (cross-linked agaroses, manufacturer: Amersham Pharmacia Biotech. Sepharose is obtainable with various linkers/spacers as well as with a variety of functional groups e.g. NHS esters, CNBr-activated, amino, carboxy, activated thiol, epoxy etc. From: Pharmacia LKB Biotechnology, Affinity Chromatographyz—Principles and Methods, Sweden 1993), Sephacryl (from: product information of Amersham Pharmacia Biotech. Spherical allyl-dextran and N,N-methylene bisacrylamide), Superdex (spherical, consisting of cross-linked agarose and dextran. From: product information of Amersham Pharmacia Biotech), trisacryl (obtained by polymerizing N-acryloyl-2-amino-2-hydroxymethyl-1,3-propanediol. From: Affinity Chromatography—a practical approach, IRL Press, Oxford, 1985), paramagnetic particles, Toyopearl™ (TosoHaas., semirigid, macroporous, spherical matrix. Manufactured from a hydrophilic vinyl copolymer. Obtainable with various functionalizations such as tresyl, epoxy, formyl, amino, carboxy etc. from: product information TosoHaas), nylon-based matrices, tentagel (Rapp polymers, from: http://www.rapp-Polymere.com/preise/tent_sum.htm. Copolymers consisting of a low cross-linked polystyrene matrix which is modified with polyethylene glycol or polyoxyethylene. The polyethylene glycol or polyoxyethylene units carry various functional groups), polystyrene.

Other matrices are for example silica gel, alumosilicates, bentonite, porous ceramics, various metal oxides, hydroxyapatite, fibroin (natural silk), alginates, carrageen, collagen and polyvinyl alcohol.

In addition functionalized or derivatized membranes or surfaces can also be used as the matrix.

Matrices are derivatized using suitable functional groups to obtain matrices that are either already pre-activated or matrices which have to be activated by adding suitable agents. Examples of non-activated functional groups that can be used to derivatize matrices are amino, thiol, carboxyl, phosphate, hydroxy groups etc. Examples of activating derivatizations of matrices are functional groups such as hydrazide, azide, aldehyde, bromoacetyl, 1,1′-carbonyldiimidazole, cyanogen bromide, epichloro-hydrin, epoxide (oxirane), N-hydroxysuccinimide and all other possible active esters, periodate, pyridyl disulfide and other mixed disulfides, tosyl chloride, tresyl chloride, vinyl sulfonyl, benzyl halogenides, isocyanates, photoreactive groups etc.

All matrices through which plasma and preferably also whole blood can be passed are particulary suitable for apheresis such as organic polymers based on for example methacrylates, natural polymers based for example on cross-linked sugar structures or also inorganic polymers based for example on glass structures (CPG, controlled pore glass). The solid phase modified with the ligands, i.e. nucleic acids and preferably functional nucleic acids, which is suitable for plasmapheresis or apheresis is filled into a housing made of glass, plastic or metal to form an apheresis device.

The individual components of an apheresis apparatus are known to a person skilled in the art. Examples of commercial apheresis systems are the liposorber system from the Kaneka Corporation, the DALI system (direct adsorption of lipids) containing the haemoadsorption instrument 4008 ADS from Fresenius AG, Bad Homburg, the H.E.L.P. system (heparin-induced extracorporeal LDL precipitation) from B. Braun AG, Melsungen the systems Ig-Therasorb, LDL Therasorb and Rheosorb from PlasmaSelect AG, Teterow, etc. (http://www.dialysis-north.de/presents/apheresetechnikshow.htm).

The nucleic acid can be bound or immobilized, directly or indirectly by the formation of covalent bonds between the matrix, preferably the solid phase, and the nucleic acid, preferably the functional nucleic acid such as an aptamer or Spiegelmer, by the formation of coordinate bonds (complexes) or by utilizing non-covalent interactions mediated by hydrogen bonds, coulombic interactions or hydrophobic interactions. Indirect coupling is understood herein to mean that the nucleic acid is or becomes bound to the matrix or matrix material which is mediated by another structure or compound which is referred to as a linker structure or spacer. The other structure can be present on the nucleic acid, on the matrix or on both. The binding of the other structure to the matrix or to the nucleic acid can be any of the types of binding described above or a combination thereof.

Use of such other structures is advantageous since the binding results in a particularly stable and specific binding of the functional nucleic acid to the matrix or to the solid phase.

In this connection a linker structure is a structure which is located between the matrix and the nucleic acid. Hence a linker structure is defined more in a functional sense than a chemical sense and mediates the binding between the nucleic acid and matrix. Such linker structures are often composed of two or more components and typically one component is bound to the nucleic acid and the other component is bound to the matrix. Examples of such linker structures are, inter alia, the biotin-avidin system (M. Wilchek, E. A: Bayer, Avidin-Biotin Technology, Methods Enzymol 1990, 184, p. 1-746), where the avidin can be replaced by suitable derivatives or analogues such as streptavidin or neutravidin. Another such linker structure is composed of fluorescein and an antibody directed against fluorescein or of digoxigenin and an antibody directed against digoxigenin.

Spacers known to persons skilled in the art especially for synthesis are another type of such structures that can be used to mediate the binding of a nucleic acid to a matrix. In contrast to a linker structure, the main function of a spacer is to set a defined distance between two structures or compounds. This is typically achieved by firstly binding the spacer to one of the two partners to be connected. However, it is also possible that a linker is bound to both binding partners. In addition the spacer, like the linker structure, also has the function of mediating binding between the binding partners.

It is obvious to a person skilled in the art that there is an overlap between linker structures and spacers. Hence it is indeed possible that linker structures serve as spacers and conversely spacers serve as linker structures.

Spacers are typically present in the form of X-spacer-Y, X-spacer-X or Y-spacer-X where the spacer results in or defines a distance between the functional groups X and/or Y, and the groups X and Y are the actual linkage between the matrix and the nucleic acid. The spacer ends up as linking atoms after the binding of the nucleic acid and matrix. However, spacers can also be part of the linker structures described above and can for example comprise a tetraethylene glycol unit (example Biotin TEG, Glen Research, Virginia, USA) or six carbon atoms (example hexamethylene diamine) or four atoms linked by an acid amide bond (example glycyl glycine).

When selecting spacers, compounds are generally preferred which are composed of at least four atoms, the atoms being selected from the group comprising C atoms and heteroatoms. The term heteroatoms is familiar to organic chemists and includes among others the following atoms: nitrogen, oxygen, silicon, sulphur, phosphorus, halogens, boron and vanadium.

The spacer preferably consists of at least four atoms which can be in a linear or branched arrangement.

When selecting or designing a suitable spacer, it is necessary that the intrinsic groups are sufficiently hydrophilic due to the fact that the coupled nucleic acid comes into contact with a hydrophilic system, in particular an aqueous system, in the typical fields of application such as affinity chromatography or apheresis. Hence spacers based on polyethylene glycol units are also preferred.

Linker structures or spacers can be used for any of the types of binding or immobilization of a nucleic acid on a matrix described herein. This means that within the scope of the invention, linker structures or spacers can be used to bind the nucleic acid via the 3′ end of the nucleic acid. If the nucleic acid is additionally immobilized on the matrix by means of at least one other site on the nucleic acid, whether via the 5′ end and/or a site within the nucleic acid sequence, this site can be in turn bound or immobilized by means of a linker structure or a spacer essentially irrespective of whether a linker structure or spacer is used at the 3′ end of the nucleic acid.

A special embodiment of a simultaneous binding of the nucleic acid via its 3′ end and its 5′ end is to use a linker structure or a spacer where both ends of the nucleic acid are joined by the linker structure or the spacer and then bound to the matrix. This results in the formation of a Y-shaped structure.

Functional nucleic acids are covalently immobilized under conditions under which covalent or non-covalent interactions between the solid phase and the non-functionalized sugar phosphate backbone, the non-functionalized sugar or the non-functionalized nucleobase are reduced as far as possible. The covalent bond can for example be an acid amide, an amine, a Schiff's base, a phosphoramidite, a phosphoric acid ester, a phosphoric acid thioester, a thioether, a thioester, a disulfide, an ether, a C—C— single or multiple bond, an oxime, a carbamate, a nitrogen single or multiple bond, an Si—C—, or S—O bond or any other covalent bond of the type X—X, X—Y, Y—X, X═X, X═Y, Y═X etc. . . .

For the covalent immobilization, either the nucleic acid to be coupled and/or the solid phase can be chemically pre-activated. The coupling then takes place by incubating the solid phase and nucleic acid in a suitable medium. Alternatively the coupling between the solid phase and nucleic acid can be carried out by adding suitable activating reagents (in situ); examples of these are CDI (1,1′-carbonyldi-imidazole, protocols in: Affinity Chromatogray, p. 42 ff.), EDC (1-ethyl-3-(3′-dimethylaminopropyl)-carbodiimide (protocols in: Pharmacia LKB Biotechnology, Affinity Chromatography—Principles and Methods, Sweden 1993, p. 39 ff) and cyanogen bromide (L. Clerici et al., Nucl. Acids. Res. 1979, 6, 247-258). The addition of homobifunctional and heterobifunctional linker molecules such as glutardialdehyde, adipic acid dihydrazide, ethylene glycol-bis[succinimidylsuccinate] or 4-N-(maleimidomethyl)cyclohexanecarboxylic acid N-hydroxysuccinimide ester, etc. (review in: Sigma product catalogue, “Biochemikalien und Reagenzien für die Life Science Forschung”, 1999, p. 303-305) can be used for covalent linkage to a solid phase. Covalently immobilized nucleic acids can also be obtained directly by solid phase synthesis in which case the linkers between the solid phase and nucleic acid have to be stable under the deprotecting conditions for the protecting groups required for the synthesis. Examples of covalent immobilization methods for nucleic acids known in the prior art are the carbodiimide-mediated immobilization of DNA on solid phases containing hydroxy groups via terminal phosphate groups to form phosphoric acid esters [P. T. Gilham, 1997, Methods in Enzymology, vol. 21, part D, 191-197], the carbodiimide-mediated immobilization of terminally phosphorylated nucleic acids on solid phases containing amino groups to form phosphoramidates [S. S. Gosh, G. F. Musso, 1987, Nucl. Acids Res., 15, 5353-5372], the immobilization of aminoalkyl-modified nucleic acids on N-hydroxysuccinimide-activated carboxyl groups to form acid amides [S. S. Gosh, G. F. Musso, 1987, Nucl. Acids Res., 15, 5353-5372], the 1,1′-carbonyldiimidazole (CDI)-mediated binding of aminoalkyl-modified nucleic acids to surfaces containing hydroxy groups to form carbamates [R. Potyrailo et al., Anal. Chem., 1998, 70, 3419-3425], the reductive coupling of amino-alkyl-modified oligonucleotides to aldehyde groups of a solid phase [E. Timofeev et al., 1996, Nucl. Acids Res., 24, 3142-3148] etc.

Nucleic acids can be non-covalently immobilized using affinity ligand-ligand interactions in which the nucleic acid is linked to one member of the interacting pair whereas the other affinity ligand is immobilized on the surface of the solid phase. Examples of affinity ligand pairs are biotin-avidin (-streptavidin, -neutravidin), antibody-antigen, nucleic acid binding protein—nucleic acid, hybridizations between complementary nucleic acids etc . . . . Non-covalent interactions can for example be used to specifically bind a biotinylated nucleic acid to a streptavidin matrix [T. S. Romig et al., 1999, Journal Chromatogr. B, 731, 275-284]. A nucleic acid provided with a poly-A tail (e.g. messenger RNA) can be bound specifically to a poly-dT matrix [J. Gielen, 1974, Arch. Biochem. Biophys. 163, 146-154]. A nucleic acid derivatized with fluorescein can be bound to a surface coated with anti-fluorescein antibodies (or vice versa).

Coordinate bonds (e.g. metal complexes) can also be used to immobilize nucleic acids on solid phases. In such systems either the nucleic acid is functionalized with a metal ion and the complex ligand is located on the surface of the solid phase, or the nucleic acid is functionalized with a complex ligand and the metal ion is located on the solid phase. An example of the immobilization of a nucleic acid utilizing coordinate bonds is the functionalization of a nucleic acid containing 6 consecutive 6-histaminyl purine units (H6-tag) and its immobilization on an Ni²⁺ matrix [M. Changee, 1996, Nucl. Acids Res., 24, 3806-3810].

Although nucleic acids coupled via their 3′ end are already very stable and in particular are very resistant to nucleases i.e. endonucleases as well as 3′ and 5′ exonucleases, this can be further increased by a 5′-terminal modification of the nucleic acid. Suitable 5′-terminal modifications are for example non-naturally configured nucleosides such as L-C, L-G, L-T, L-A or an inverse T or carbon chain of any length or a PEG modification or any other chemical modification which is not a substrate for a naturally occurring enzyme.

As already described above, it also surprisingly turned out that nucleic acids and functionalized nucleic acids (aptamers and Spiegelmers) can in some cases be sterilized by steam at 120° C. for at least 30 minutes when they have been immobilized by the immobilization strategies according to the invention on a matrix and above all on a solid phase. This unexpected property of specifically immobilized aptamers or Spiegelmers opens up completely new perspectives for the production process of adsorbers based on functional nucleic acids such as aptamer adsorbers or Spiegelmer adsorbers. Hence conventional reaction steps can be used to modify the support material with the respective nucleic acid as the ligand, fill the modified support material into an appropriate device and sterilize the entire adsorber by steam and subsequently seal it in sterile packs. It is then suitable for a therapeutic application in extracorporeal blood cleansing.

The method disclosed herein for eluting target molecules from immobilized functional nucleic acids has a number of advantages compared to the methods in the prior art. In particular it is not necessary to use large amounts of salts such as 8 M urea which would result in a considerable salt load especially in industrial applications. Also other compounds that may be difficult to dispose of that are used in the prior art elution methods such as guanidinium thiocyanate are also not present. The elution method described herein can be used to immobilize nucleic acids for apheresis as well as for an affinity medium, for example for affinity purification such as affinity chromatography. Immobilized functional nucleic acids can for example be used in an affinity purification to purify recombinant proteins. Another application of immobilized nucleic acids according to the invention is in the field of diagnostics in which the disclosed immobilized nucleic acids are used in particular as an affinity medium.

Use of desalted water such as distilled water and in particular twice distilled water for elution purposes is surprising since particular emphasis is made on the design of specific buffers with distinct salt concentrations in conventional elution techniques. In the present case it appears that the absence of such salts in the system comprising target molecule and nucleic acid alters the folding of the nucleic acid to such an extent that there is no longer a high binding constant to the target molecule. In this connection it is particularly noteworthy that the nucleic acid can be renatured at any time and hence the use of this specific elution method does not stand in the way of a regeneration of the matrix carrying the Spiegelmer.

With regard to the elevated temperatures at which this elution method is carried out, temperatures of at least 45° C. are preferred. Higher temperatures such as at least 50° C. or at least 55° C. are thus preferred. When desalted water is used for the elution, the choice of temperature essentially depends on the complex that is actually present of immobilized nucleic acid and target molecule.

Alternatively the target molecule bound to the immobilized nucleic acid can also be eluted using the elution methods known in the prior art. These include among others the so-called denaturing elution. Denaturing elution typically uses one of the following compounds preferably under high molar conditions: guanidinium thiocyanate, urea, guanidinium hydrochloride, EDTA (ethylene diamine tetraacetate) sodium hydroxide and potassium hydroxide. Typical molarities in case of the use of guanidinium thiocyanate or guanidinium hydrochloride are 4 M, and 8 M if urea is used. Basic compounds such as NaOH or KOH can also be used for the denaturing elution. Furthermore it is generally possible to use high salt concentrations involving Na ions or potassium ions.

The invention is further illustrated on the basis of the figures, examples and the sequence protocol from which other features, embodiments and advantages of the invention may be derived.

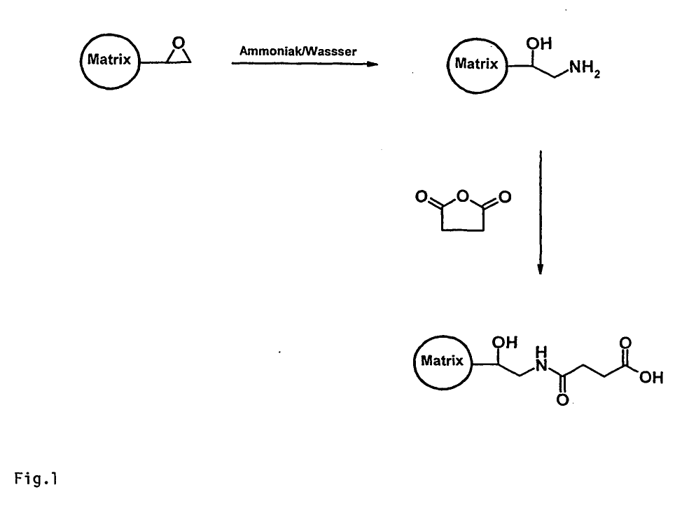

FIG. 1 shows the process of covalently modifying a solid phase in which a matrix containing oxirane groups is firstly converted into a matrix containing primary amino groups. A pre-activated, bifunctional linker is then used to introduce a carboxylic acid function on the matrix;

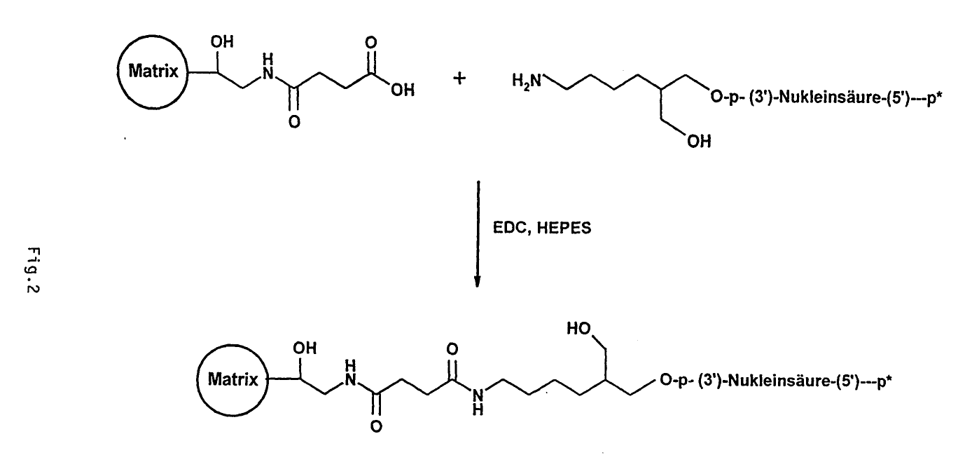

FIG. 2 shows the binding of a 3′-aminoalkyl-modified, 5′-terminal radioactively-labelled nucleic acid on the solid phase using a coupling reagent;

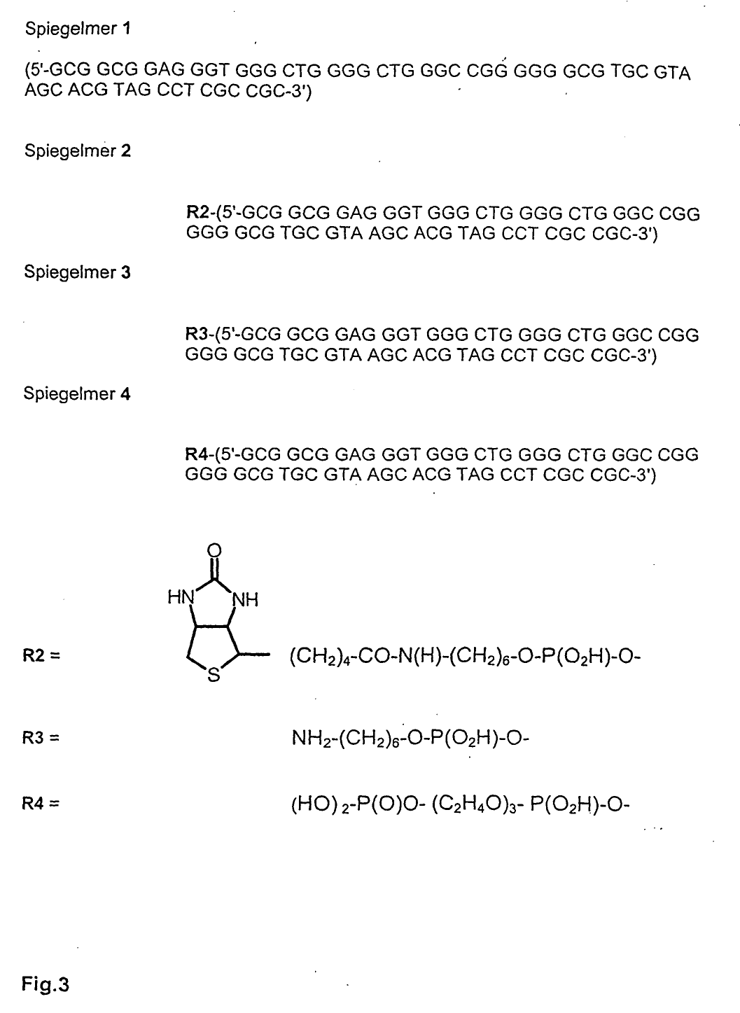

FIG. 3 shows an overview of the various derivatives of the immobilized nucleic acids as described in the examples;

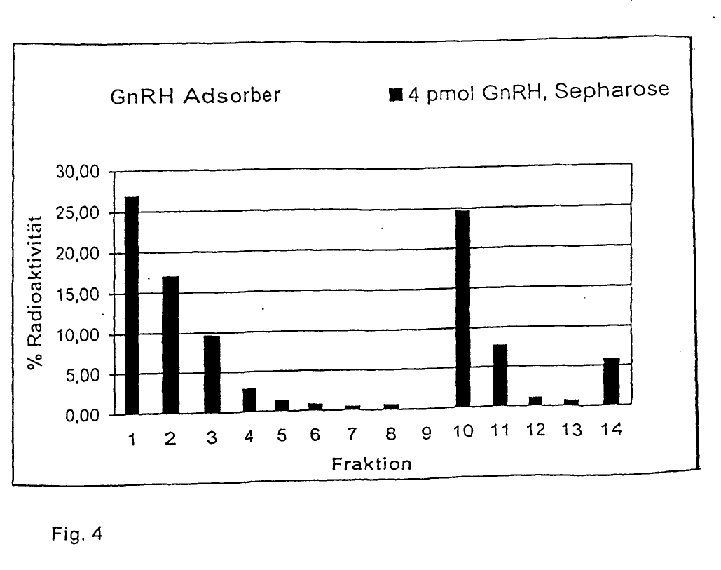

FIG. 4 shows the adsorption profile of a GnRH adsorber based on a Sepharose matrix;

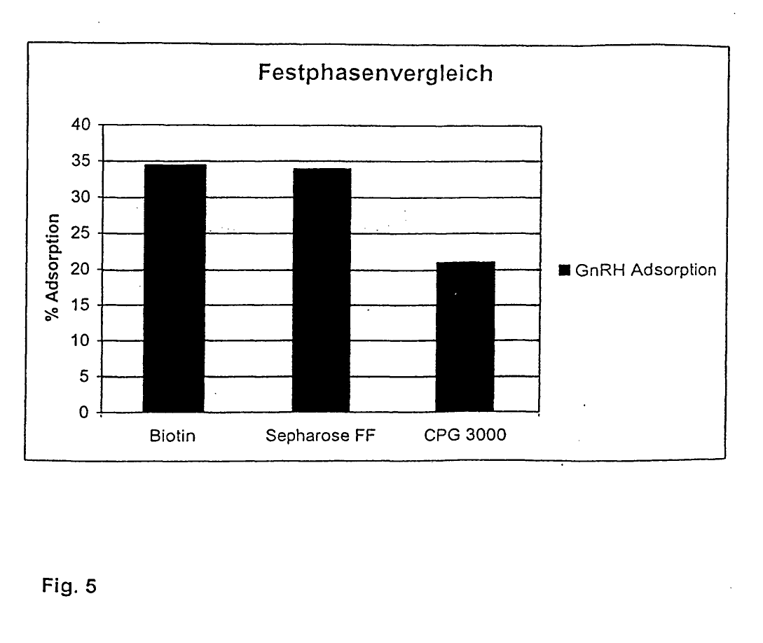

FIG. 5 shows the adsorption capacity of various adsorbers consisting of various matrix materials;

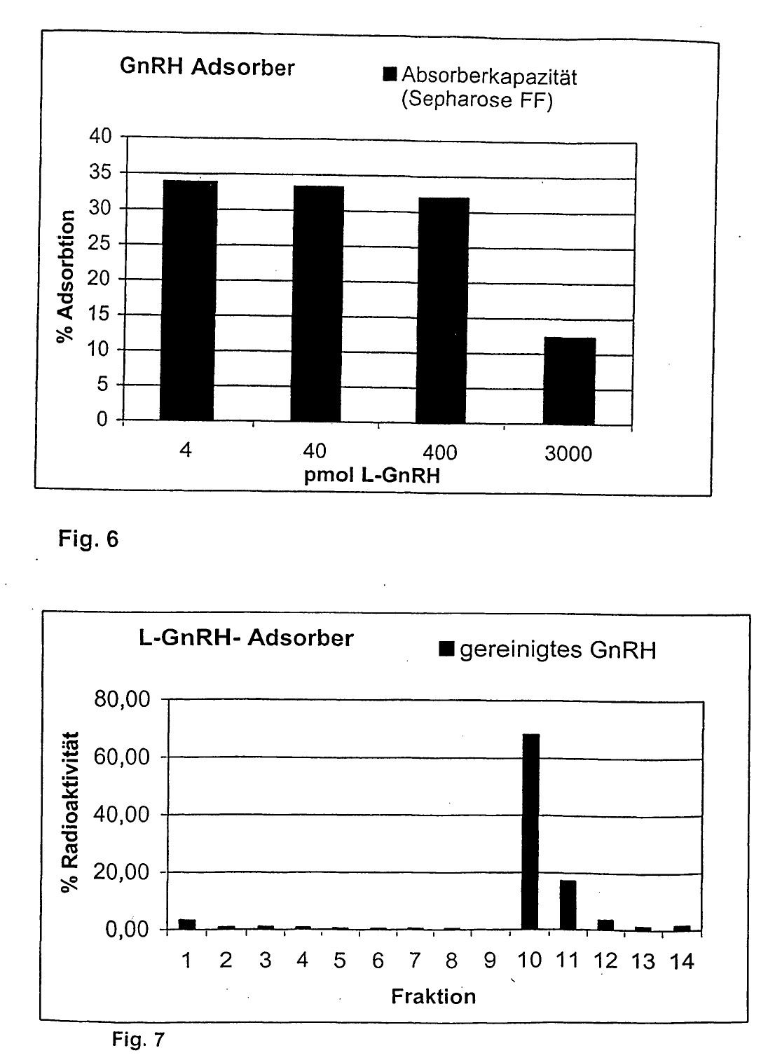

FIG. 6 shows the adsorber capacity;

FIG. 7 shows the adsorption profile of a GNRH adsorber based on a Sepharose FF matrix and

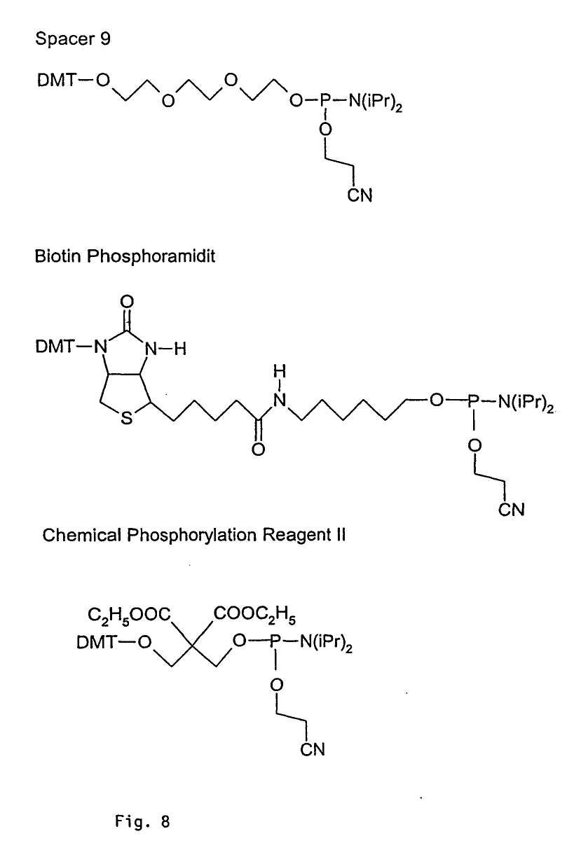

FIG. 8 shows the chemical structural formulae of spacer 9, biotin phosphoramidite and chemical phosphorylation reagent II.

### EXAMPLES

### Example 1

### Immobilization of Radioactively Labelled Amino-Modified DNA on Eupergit C and Subsequent Investigation of the Stability

The application example demonstrates the covalent modification of a solid phase, the subsequent coupling or binding of amino-modified DNA as shown in FIG. 2 and the examination of the stability of the bound oligonucleotides.

100 mg Eupergit C 250L (Röhm Pharma GmbH, Weiterstadt is used. Copolymer of methacrylamide, N-methylene-bis-methacrylamide and monomers containing oxirane groups. Macroporous beads of 250 μm diameter. Biocompatible: exhibits no toxic reactions at all when administered orally to rats: the oral LD50 is >15 g/kg. Source: product information Röhm Pharma) was treated overnight at room temperature with 3 ml of a 16% ammonia solution. Subsequently the solid phase was washed 5× with 3 ml distilled water each time and 5× with 3 ml freshly distilled pyridine each time. It was treated overnight with 3 ml of a saturated solution of succinic anhydride in pyridine. After completion of the reaction, the solid phase was washed 5× with 3 ml distilled water each time and 5× with 3 ml 0.1 M HEPES buffer (pH 7.5) each time.

A freshly prepared solution of 100 μl 0.1 M HEPES (4-(2-hydroxyethyl)-piperazine-1-ethanesulfonic acid) containing 0.2 M EDC (1-ethyl-3-(3′-dimethylaminopropyl)-carbodiimide) was added to 20 mg of the carboxyl-modified solid phase and thoroughly mixed. The suspension was admixed with 1 nmol 3′-aminomodified and 5′-phosphorylated (³²P) DNA oligonucleotide which had been prepared on a state of the art synthesizer using phosphoramidite chemistry (Blackburn, G. M. and Gait, M. J., 1992, Nucleic acids in Chemistry and Biology, IRL Press Oxford). The 5′ end of the DNA oligonucleotide was labelled with radioactive phosphate with the aid of the enzyme T4 polynucleotide kinase and [γ-³²P]-adenosine triphosphate after incubation for one hour at 37° C. in 50 mM Tris/HCl, pH 7.6, 10 mM magnesium chloride, 5 mM 1,4 dithiothreitol, 100 μm spermidine and 100 μM EDTA. The labelled and subsequently purified DNA molecule was then incubated for 3 hours at room temperature while carefully mixing the reactants listed above. After completion of the reaction, the solid phase was washed 10× with 1 ml distilled water each time and the solvent was removed. The yield of the immobilization was determined by Cerenkov counting. In order to determine the extent of non-covalent binding to the affinity matrix, parallel experiments were carried out under the described conditions in the absence of EDC. The result are shown in table 1.

TABLE 1
Results of immobilizing 3′-aminoalkyl-modified nucleic acids
radioactively labelled at the 5′ terminus to a carboxyl-modified
solid phase and of a control experiment (without EDC). In addition
the results of steam sterilization of covalently bound nucleic acids
are shown.

| | DNA + EDC | DNA − EDC | RNA + EDC | RNA − EDC |
|---|---|---|---|---|
| | [%] | [%] | [%] | [%] |
| immobilization | 95 ± 3 | 15 ± 13 | 94 ± 2 | 23 ± 4 |
| sterilization | 88 ± 3 | 6 ± 3 | 85 ± 5 | 10 ± 5 |

Table 1 shows the yield based on the amount of nucleic acid used of the covalent immobilization of DNA and RNA on modified Eupergit C 250L. The amount of immobilized nucleic acid in the absence of the coupling reagent EDC (−EDC) shows the extent of non-covalent unspecific coupling to the matrix. The second line shows the result of a single steam sterilization. In the case of covalently immobilized DNA, about 88% remains on the matrix after steam sterilization whereas only 6% of the non-covalently bound DNA remains on the matrix. This result demonstrates the stabilizing effect of the immobilization. Corresponding results are shown for the immobilization and steam sterilization of 3′-aminoalkyl-modified RNA.

Since the nucleic acids are radioactively labelled at the 5′ terminus and the process of detachment from the matrix was determined by Cerenkov counting, the detected molecules are intact nucleic acids without degradation due to strand breaks.

The steam sterilization of the affinity matrix was carried out in a laboratory autoclave. The sterilization was carried out for 20 min at 121° C. and 2.4 bar. The matrix remained in the autoclave for a total period of 2 h. The amount of oligonucleotides cleaved during autoclaving was determined by Cerenkov counting (see table 1 for results).

The stability of the oligonucleotides was examined by incubating the solid phase in human serum at 37° C. The pH value of the serum was additionally buffered by adding 10 mM sodium phosphate, pH 7.0. Similar stability studies were carried out using plasma instead of serum. The results of these studies are shown in tables 2 and 3 in which the stated values represent the loss of oligonucleotides expressed in % of the originally immobilized amount. The length of the immobilized nucleic acid was 52 (52 mer). It was immobilized by means of a biotin residue present at the 3′ end of the nucleic acid ((52 mer) 3′ biotin) and the nucleic acid had an L-nucleotide at the 5′ end for a further experiment (5′-L (52 mer) 3′ biotin).

TABLE 2
Stability of the immobilized nucleic acid when exposed to plasma

| | loss of immobilized nucleic acid (in %) | | | |
|---|---|---|---|---|
| | after incubation in plasma for | | | |
| oligonucleotide | 3 h | 5 h* | 24 h | half-life in h |
| (52mer) 3′ | 9 | 12 | 40 | 27 |
| biotin | | | | |
| 5′-L (52mer) 3′ | 7 | 9 | 34 | 35 |
| biotin | | | | |

*calculated value from the corresponding half-life

TABLE 3
Stability of the immobilized nucleic acid when exposed to serum

| | loss of immobilized nucleic acid (in %) | | | |
|---|---|---|---|---|
| | after incubation in plasma for | | | |
| oligonucleotide | 1.5 h | 5 h* | 23 h | half-life in h |
| (52mer) 3′ | 5 | 8 | 33 | 40 |
| biotin | | | | |
| 5′-L (52mer) 3′ | 2 | 6 | 25 | 59 |
| biotin | | | | |

*calculated value from the corresponding half-life

The tables show that the amount of radioactivity on the solid phase was still more than 80% even after several hours. Non-immobilized oligonucleotides are degraded by more than 50%.

### Example 2

### Immobilization of Radioactively Labelled Amino-Modified RNA on Eupergit C and Steam Sterilization

The experiment was carried out as described in example 1 using a 3′ amino-modified and 5′ phosphorylated (³²P) RNA oligonucleotide. RNA oligonucleotides were synthesized by conventional prior art phosphoramidite methods and subsequently purified (Ogilvie, K. K. Usman, M., Nicoghosian, K. and Cedergren, R. J. Proc. Natl. Acad. Sci. USA, vol. 85, 5764-5768). 5′ labelling with ³²P was carried out similarly to the DNA labelling described above. The results are summarized in table 1 above.

### Example 3

### Synthesis of ³H-GnRH and Oligonucleotides

L-GnRH (Pyr-His-Trp-Ser-Tyr-Gly-Leu-Arg-Pro-Gly-NH₂ (SEQ ID NO:1)) having a purity of >90% was purchased from Jerini Bio Tools, Berlin, Germany. The radioactive labelling was performed by Amersham Pharmacia Biotech by halogenating the tyrosine residue followed by reductive dehalogenation with tritium gas. According to the manufacturer the radiochemical purity of the ³H-GnRH preparation was 45%. The tritium-labelled peptide was not purified further. The statement that the radiochemical purity is 45% means that 45% of the radioactivity is located in the GnRH, the remaining 55% radioactivity is bound to other components of the GnRH preparation.

The GnRH-binding DNA Spiegelmer 1 (cf FIG. 3) having the sequence
5′-GCG GCG GAG GGT GGG CTG GGG CTG GGC CGG GGG GCG TGC GTA AGC ACG TAG CCT CGC CGC-3′ (SEQ ID NO.2)
   was prepared on an Akta Pilot 10 synthesizer, Amersham Pharmacia Biotech using standard phosphoramidite chemistry on a 20 μmol scale. The L-DNA phosphor-amidites were purchased from ChemGenes. The various 5′ modifications i.e. biotin, amino and phosphate group, were added to the oligonucleotide 1 as shown in FIG. 3 by continuing the above-mentioned synthesis on a 1 μmol scale on an ABI 394 DNA synthesizer, Applied Biosystem using the appropriate phosphoramidites.

5′-biotinylated DNA Spiegelmer 2 was prepared by reaction with 5′-biotin-phosphoramidite([1-N-(dimethoxytrityl-biotinyl-6-aminohexyl]-(2-cyanoethyl)-(N,N-diisopropyl)-phosphoramidite), Glen Research followed by deprotection and purification by PAGE (10% PAA/8 M urea). 5′-Aminohexyl-modified DNA Spiegelmer 3 was prepared by treating 1 with 5′-amino modifier C6-phosphoramidite(N-monomethoxytrityl-aminohexyl-[(2-cyanoethyl)-(N,N-diisopropyl)]phosphoramidite, Glen Research and 5′-phosphorylated DNA Spiegelmer 4 was prepared by firstly reacting Spiegelmer 1 with spacer 9 phosphoramidite(9-O-dimethoxytrityl-triethylene glycol, 1-[(2-cyanoethyl)-(N,N-diisopropyl)]-phosphoramidite), Glen Research, followed by coupling with chemical phosphorylation reagent II ([3-(dimethoxytrityloxy)2,2-dicarboxyethyl]-propyl-(2-cyanoethyl)-(N,N-diisopropyl)-phosphoramidite), Glen Research. The DNA Spiegelmers 3 and 4 were MMT-/DMT-ON purified by preparative RP-HPLC on SOURCE 15 RPC, Amersham Pharmacia Biotech. Finally Spiegelmers 2 to 4 were desalted on an NAP-10 column, Amersham Pharmacia Biotech.

### Example 4

### Preparation of the Affinity Columns

The biotinylated Spiegelmer 2 (9.4 nmol) was immobilized at room temperature on neutravidin agarose (Pierce, 500 ml gel, immobilized NeutrAvidin, Pierce; capacity 20 units biotin-PNP ester/ml gel) in buffer A (100 μl, 20 mM Tris HCl, pH 7.4, 137 mM NaCl, 5 mM KCl, 1 mM MgCl₂, 2 mM CaCl₂, 0.005% Triton X-100). Room temperature is understood herein as a temperature range of about 22 to about 25° C. After 45 minutes the immobilization yield, which was estimated on the basis of the UV absorbance (260 nm) of the applied and of the unbound Spiegelmer, was almost quantitative.

In order to immobilize Spiegelmer 3 on Sepharose, the NHS-activated Sepharose 4 Fast Flow (Amersham Pharmacia Biotech, 1 ml, 16-23 μmol NHS/ml dehydrated medium) was firstly washed with 15 column volumes (CV) of 1 mM hydrochloric acid. After incubation for 1 h at room temperature with Spiegelmer 3 (20 nmol) in 0.3 M NaHCO₃ buffer (pH 8.5, 200 μl), the matrix was rewashed with 10 mM NaOH/2 M NaCl solution (5 CV), 0.3 M NaOAc buffer (pH 5.5, 5 CV) and buffer A (5 CV). The estimated yield was 80-90%.

Spiegelmer 4 was immobilized on CPG. Before the immobilization of Spiegelmer 4 on CPG, the 3000 A lcaa CPG (long chain aminoalkyl controlled pore glass, CPG Biotech; 1 g, capacity 32.6 μmol/g) was incubated overnight at room temperature with 0.1% herring sperm DNA (Roche) in buffer B (10 ml, 10 mM Tris-HCl/1 mM EDTA, pH 7.4). After washing 0.6 ml matrix with buffer B (5 ml) and 0.2 M N-methyl-imidazole hydrochloride (2 ml, pH 6), the CPG was shaken overnight at room temperature with a solution of DNA Spiegelmer 4 (18.3 nmol) in 0.1 M N-methyl-imidazole hydrochloride/0.1 M EDC (0.5 ml, pH 6). The estimated coupling yield was 40-50%.

The affinity media prepared in this manner are those where a GnRH-binding DNA Spiegelmer is bound covalently as well as those where the binding of the Spiegelmer is non-covalent. Said Spiegelmer was immobilized via a 5′ modification on neutravidin agarose, NHS Sepharose 4 Fast Flow and 3000A lcaa-CPG. The non-covalent immobilization of the 5′-biotinylated Spiegelmer 2 on neutravidin agarose was almost quantitative. The covalent binding of the DNA Spiegelmer to carboxyl Sepharose and lcaa-CPG was 80-90% and 40-50% respectively via an amide or phosphoramidite binding.

### Example 5

### Affinity Chromatography

In order to characterize the absorption properties of the matrices derivatized with Spiegelmers, 100 μl of the corresponding matrix was filled into a 800 μl Mobicol column (MoBiTec). The column outlet was closed with a stopper and a solution of GnRH (500,000 cpm, ca. 4 pmol) in buffer A (100 μl) was added to the column. The mixture was shaken for 30 minutes at room temperature. The corresponding non-derivatized matrices (100 μl) were incubated as a control under the same conditions using the same amount of peptide. After incubation the eluate was collected and the columns were washed with buffer A (7×100 μl). The amount of ³H-labelled GnRH in the eluate and the wash fractions of all fractions were quantified using an LS 5000 scintillation counter (Beckman). The amount of radioactivity was very close to the background value after washing 7 times. The bound peptide was eluted from the solid phase by incubation with 200 μl 4 M guanidinium thiocyanate for 15 minutes at 55° C. The elution process was repeated once with 200 μl 8 M urea for 15 minutes at 55° C. and washing twice with 100 μl 8 M urea.

The following table 1 shows the absorption properties of various (Spiegelmer) matrices expressed as % radioactivity. The symbol (x) denotes that the matrix is one which carries the D-enantiomer of the Spiegelmer as the ligand and consequently the aptamer. All data assume a radiochemical purity of 45%.

TABLE 1

| | | | | | | unmodified |
|---|---|---|---|---|---|---|
| | Spiegelmer | Sepharose | Spiegelmer | unmodified | Spiegelmer- | CPG |
| matrix | Sepharose | (x) | agarose | agarose | CPG 3000 | 3000 |
| retained | 40 | 4.2 | 36.5 | 1.0 | 23 | 2 |
| eluted | 34 | 0.8 | 34.5 | 0.8 | 20.7 | 0.8 |
| background | 6 | 3.4 | 2 | 0.2 | 2.3 | 1.2 |
| binding | | | | | | |

36.5% of the radioactivity was retained on the derivatized neutravidin matrix with a background binding of radioactivity of 2% which does not bind to the non-derivatized matrix. If one assumes a radiochemical purity of 45%, this means that in the present case ca. 81% of the radioactivity located on GnRH was bound. After denaturation of the GnRH-binding Spiegelmer, 95% of the retained radioactivity was eluted from the matrix. The derivatized carboxyl Sepharose exhibits similar properties in which a background binding of 6% was observed with a retained radioactivity of 40% corresponding to 89% of GnRH. 85% of this is in turn recovered after denaturation. The result is also shown in FIG. 4 where fraction 1 represents the flow through, fractions 2 to 8 are wash fractions, fractions 10 to 13 are the fractions which elute under denaturation and fraction 14 is the fraction containing the solid phase. In contrast less radioactivity i.e. 23% was retained by the CPG matrix which had a background binding of 2%.

FIG. 5 also shows a comparison of the absorption properties of various matrix materials having the Spiegelmer of SEQ ID NO: 1 as the ligand.

In order to differentiate between unspecific binding to the non-derivatized support and unspecific binding to the oligonucleotide, the D-enantiomer of the Spiegelmer which exhibits no interaction with GnRH was immobilized on NHS Sepharose 4 Fast Flow in a similar manner to the Spiegelmers. After incubation only 4.2% of the radioactivity was retained on the matrix and 0.8% was eluted after denaturation of the oligonucleotide.

### Example 6

### Determination of the Matrix Capacity

A Sepharose matrix loaded with 1.7 nmol Spiegelmer/100 μl phase (matrix) was incubated with various amounts of GnRH which had been admixed with 0.1 μl of the crude preparation of ³H-GnRH (45% purity). The matrix was washed and the bound materila was eluted as described in example 5.

The capacity of the Sepharose matrix was determined by incubation with an excess of GnRH which had been admixed with the crude preparation of ³H-GnRH. The application of 3 nmol GnRH to the matrix showed that 12.4% of the radioactivity was bound which corresponds to 28% when the radiochemical purity of 45% of the GnRH preparation is taken into consideration. With a loading of 1.7 nmol Spiegelmer, 840 pmol GnRH was absorbed. The incubation with less GnRH (4.40 and 400 pmol) clearly demonstrated an almost quantitative adsorption of the peptide as also shown in FIG. 6 and also in the following table 2. This is the surprising proof that the oligonucleotide molecule retains its binding properties during and after immobilization.

TABLE 2
Adsorber capacity of GnRH Spiegelmer Sepharose

| pmol GnRH | % eluted |
|---|---|
| 4 | 34 |
| 40 | 33.4 |
| 400 | 32 |
| 3000 | 12.4 |

### Example 7

### Characterization of the Bound Fraction

The neutravidin matrix was incubated with the crude ³H-GnRH preparation and, as described in the previous examples, washed with water. The bound material was eluted by incubating twice with redistilled water (0.2 ml) for 15 minutes at 55° C. The combined eluates were lyophilized and characterized by their binding to a GNRH-specific antibody. This successfully validated the result of a binding using the immobilized Spiegelmer by the established system in the form of a GnRH-specific antibody (obtained from Biotrend, Cologne, Germany).

### Example 8

### Equilibrium Dialysis

An equilibrium dialysis of 40 μl of 10 μM solutions of the GnRH-binding Spiegelmer in buffer A was carried out against 40 μl solutions purified GnRH (of about 20,000 cpm) in buffer A or of unpurified GnRH in buffer A in microdialysis chambers. The two compartments were separated by a Spectra/Por (Spektrum) cellulose ester dialysis membrane (molecular exclusion limit 8-10,000 Dalton). After incubation for 24 h at room temperature, 35 μl aliquots were removed from each compartment and the radioactivity was determined by Cerenkov scintillation counting. The difference of the radioactivity in the two compartments relative to the radioactivity present in the compartment containing the Spiegelmer was calculated as a percentage of the GnRH bound to the Spiegelmer or to the antibody.

It was found that after two purification steps 87±2% of the purified GNRH had bound to the Spiegelmer at DNA saturation concentrations compared with 41% of the non-purified GnRH fraction under the same conditions i.e. using the Spiegelmer. The same results were achieved with the antibody described in example 7.

### Example 9

### Use of the GnRH-Binding Spiegelmer as a Sensor

The goal was to use the GnRH-binding Spiegelmer as a sensor to test the purity of the GnRH eluted from the Spiegelmer column. In order to use the equilibrium dialysis method to check the GnRH purity, it is necessary to use milder elution conditions than in the case of denaturing guanidinium thiocyanate. A double incubation of the Spiegelmer-neutravidin column with ddH₂O, i.e. distilled water for 15 minutes at 55° C. proved to be just as effective as elution with guanidinium thiocyanate. This elution method allows an elution where the matrix containing the Spiegelmer that binds the target molecule can be recycled. This specific elution method was successfully used in further experiments even with Sephadex as the matrix.

As an additional proof, twice purified GnRH was admixed with 4 pmol non-labelled GnRH and incubated on the Spiegelmer matrix as described in the examples. In this case 89% of the added radioactivity was retained and, under denaturation, eluted from the Spiegelmer-Sepharose column. 73% was retained when using the derivatized CPG 3000 matrix. The result is also shown in FIG. 7, where fraction 1 represents the flow through, fractions 2 to 8 represent the wash fractions and fractions 10 to 13 represent the elution under denaturation. The value of fraction 14 is that of the solid phase.

With regard to the use of functional nucleic acids, in particular Spiegelmers, it can thus be stated that covalent as well as non-covalent binding to matrices is possible and hence it can be used as an affinity matrix. There is basically no difference between the adsorption behaviour of non-covalently (neutravidin agarose) and covalently (Sepharose) immobilized Spiegelmer. The binding study of L-DNA with an unpurified ³H-GnRH preparation showed that a maximum of 41% of the mixture binds at high Spiegelmer concentrations (up to 10 μM). In contrast only 34% of the mixture was retained on the Spiegelmer-neutravidin and the Spiegelmer-Sepharose column. This shows that more than 80% of the GnRH can be adsorbed. The determination of the capacity shows that 50% of the immobilized Spiegelmer is correctly folded and binds GNRH (see example 6).

The covalently immobilized L-DNA ligands disclosed herein represent the first example of a nucleic acid-based affinity matrix which is highly stable in biological liquids. The corresponding systems of the prior art do not have this biological stability.

The disclosure of the various literature references cited herein is herewith incorporated by means of reference.

The features of the invention described in the previous description, figures and sequence protocol and the claims can be individually essential as well as in any combinations for the realization of the invention in its various embodiments.

1-35. (canceled)

36. A method for apheresis or extracorporeal blood treatment comprising the step of exposing blood to a functional nucleic acid immobilized on a matrix, wherein the functional nucleic acid is a Spiegelmer.

37. The method of claim 36, wherein said nucleic acid is bound to said matrix by the 3′ terminus of said nucleic acid.

38. The method of claim 36, wherein said nucleic acid is bound to said matrix by the 5′ terminus of said nucleic acid.

39. The method of any one claims 36-38, wherein said nucleic acid is bound to said matrix via at least two sites of said nucleic acid.

40. The method of claim 36 wherein said nucleic acid comprises nucleotides selected from the group consisting of D-nucleotides, L-nucleotides, modified D-nucleotides, modified L-nucleotides and mixtures thereof.

41. The method of claim 36, wherein said nucleic acid is bound directly to said matrix.

42. The method of claim 36, wherein said nucleic acid is bound to a linker which is bound to said matrix.

43. The method of claim 42, wherein said linker comprises at least four atoms.

44. The method of claims 36, wherein said nucleic acid is bound to said matrix by a sugar moiety of the sugar phosphate backbone, a phosphate moiety of the sugar phosphate backbone or a base moiety of the nucleotides forming said nucleic acid.

45. The method of claim 36, wherein said nucleic acid is immobilized by covalent binding, non-covalent binding, hydrogen bonding, van der Waals interactions, coulombic interaction, hydrophobic interaction, coordinate binding or combinations thereof.

46. The method of claim 36, wherein said matrix is a solid phase.

47. The method of claim 46, wherein said matrix comprises an organic polymer, an inorganic polymer or both.

48. The method of claim 36, wherein said nucleic acid is at least 15 nucleotides in size.

49. The method of claim 48, wherein said nucleic acid is at least 20 nucleotides in length.

50. The method of claim 49, wherein said nucleic acid is at least 25 nucleotides in length.

51. The method of claim 50, wherein said nucleic acid is at least 30 nucleotides in length.

52. The method of claim 51, wherein said nucleic acid is at least 35 nucleotides in length.

53. The method of claim 46, wherein said solid phase is selected from the group consisting of controlled pore glass, clay, cellulose, dextran, acrylics, agarose and polystyrene.

54. The method of claim 36, wherein said nucleic acid comprises SEQ ID NO:2.

55. An apheresis device comprising a functional nucleic acid, wherein said nucleic acid is a Spiegelmer.

56. A method for producing an immobilized functional nucleic acid, wherein said functional nucleic acid is a Spiegelmer, comprising:
a) providing a nucleic acid and a matrix; and
b) reacting said nucleic acid with said matrix to form a bond between the 3′ end, the 5′ end or both of said nucleic acid and said matrix.

57. The method of claim 56, further comprising modifying the 5′ end of the functional nucleic acid before the reacting step b).

58. The method of claims 56 or 57, wherein said nucleic acid, said matrix or both are activated before reacting said nucleic acid and said matrix.

59. The method of claim 56, wherein said nucleic acid of step a) comprises a linker.

60. The method of claim 56, wherein said matrix of step a) comprises a linker.

61. The method of claim 36, further comprising eluting a target molecule bound to said immobilized nucleic acid, wherein said eluting comprises contacting said blood-exposed matrix to distilled water at an elevated temperature.

62. The method of claim 61, wherein said elevated temperature is at least 45° C.

63. The method of claim 62, wherein said elevated temperature is at least 50° C.

64. The method of claim 63, wherein said elevated temperature is at least 55° C.

65. The method of claim 36, further comprising eluting a target molecule bound to said immobilized nucleic acid, wherein said eluting comprises contacting said blood-exposed matrix to a denaturing solution.

66. The method of claim 65, wherein said denaturing solution is guanidinium thiocyanate, urea, guanidinium hydrochloride, ethylene diamine tetraacetate, sodium hydroxide or potassium hydroxide.
